(19) United States
(12) Patent Application Publication
(10) Pub. No.: US 2006/0173530 A1
(43) Pub. Date: Aug. 3, 2006

(54) Flexible cells for interconnecting stent components

(76) Inventor: Gladwin S. Das, Arden Hills, MN (US)

Correspondence Address: KONDZELLA AND CYR, PONDVIEW PLAZA, SUITE 114, 5850 OPUS PARKWAY, MINNETONKA, MN, 55343, US

(21) Appl. No.: 11/045,927

(22) Filed: Jan. 28, 2005

(51) Int. Cl. A61F 2/90

(52) U.S. Cl. 623/1.16

(57) ABSTRACT

Axially flexible stents for tracking through tortuous segments of tubular structures in body passages are disclosed. The stents, which utilize flexible interconnects, conform to such tortuous segments, track easily, and have sufficient surface areas to prevent gaps between stent components. In a preferred embodiment the interconnects have the form of “closed cells”. However, in contrast to stents of a closed cell design, the interconnects do not expand upon radial expansion of the stent during deployment. Instead, the interconnects can expand or contract axially to enable negotiating twists and turns encountered in body passageways. If the stent encounters a concavity in a body passageway, the interconnects contract. If the stent encounters a convexity, the interconnects expand.

### BACKGROUND OF THE INVENTION

1. Field of the Invention

The present invention relates to medical devices and, more particularly, to stents that can be expanded for purposes of deployment.

2. Background of the Related Art

Under normal circumstances, the heart functions as a pump to perfuse blood throughout the body through arteries. The arteries of some patients are subject to stenosis, a localized partial blockage, which narrows the passageway and interferes with normal blood flow. This condition is termed atherosclerotic coronary artery disease. It is a leading cause of morbidity in adults in the western world. One corrective procedure used to treat this disease is coronary bypass surgery, which is a highly invasive operation. In recent years a corrective procedure, percutaneous transluminal coronary angioplasty, and devices known as balloon angioplasty catheters have been widely used to correct stenotic conditions within arteries, particularly coronary arteries, in a relatively efficient manner.

An angioplasty procedure generally includes inserting a deflated balloon, mounted on a catheter, within the affected vessel or artery at the point of a stenosis. The balloon is then inflated to physically force the dilation of the partially occluded vessel. Roughly 300,000 patients per year in the United States are presently undergoing coronary angioplasty procedures. However, a substantial percentage of patients who have had balloon angioplasty redevelop the stenosis in a relatively short period of time. The reoccurrence typically becomes evident within less than about 6 months after angioplasty and may affect 30 to 40 percent of patients. The percentage of patients who have reoccurring stenoses is generally reduced by installing a “scaffolding” device, known as a stent, at the site of the stenosis. The underlying mechanism for the benefit of stenting may be as simple as preventing immediate elastic recoil and maintaining a large luminal cross-section for a few days after angioplasty. The drawbacks of stenting are thought to relate to an increased potential for thrombus formation and hyperplasia induced by metallic or other stent materials.

While coronary and other arterial stenoses are common applications for stenting, stents can also be used to treat narrowings in any hollow or tubular organs, such as the esophagus, urethra, biliary tract, and the like.

Stents are generally tubular devices, frequently made of a thin-walled metallic or woven material. They have a pattern of apertures, openings or holes defined around the circumference of the stent along most of its length. Stents may be constructed from a variety of materials, such as stainless steel, Elgiloy, nitinol, shape memory polymers, and the like. They may be formed by a variety of methods. For example, a stent may be formed by etching or cutting the stent pattern from a tube or section of stent material; or a sheet of stent material may be cut or etched according to a desired stent pattern, whereupon the sheet may be rolled or otherwise formed into the desired tubular or bifurcated tubular shape of the stent; or one or more wires or ribbons of stent material may be braided or otherwise formed into the desired shape and pattern.

Stents are typically provided in two configurations, self-expanding and balloon expandable, or hybrids thereof. Self-expanding stents are generally spring-like devices which are inserted in the body passageway in a contracted state within a delivery catheter or introducer. A self-expanding stent is biased so as to expand upon release from the delivery catheter. When released, the stent reconfigures from a contracted to an expanded state. The self-expanding stent tends to increase to a final diameter dependent on the size and configuration of the stent and the elasticity of the body passageway. Self-expanding stents expand into place when unconstrained, without requiring assistance from a balloon. Balloon expandable stents require mounting over a balloon, positioning, and inflation of the balloon to expand the stent radially outward. Generally, a balloon expandable stent will include a balloon positioned within its central passage. Once the balloon expandable stent has been properly positioned, the balloon is inflated, thereby expanding the stent so that the stent is urged in place against the body passageway. As indicated above, the amount of force applied is at least that necessary to maintain the patency of the body passageway. Once the stent is properly expanded, the balloon is deflated and withdrawn from the patient. Ideally, the stent will remain in place and maintain the target area of the body passageway substantially open to preserve a desired degree of the passage's function. Some stents may be characterized as hybrid stents which have some characteristics of both self-expandable and balloon expandable stents.

The balloon expandable stent, self-expanding stent, or a hybrid thereof may be delivered to the target area of the body passageway by a catheter system. In such systems, the catheter or introducer typically enters the body from an access location outside the body, such as through the patient's skin, or by a “cut down” technique in which the entry blood vessel is exposed by minor surgical means. Accordingly, such systems are typically considered to be minimally invasive.

In one exemplary technique, a stent may be implanted endoluminally in a blood vessel at the site of a stenosis or aneurysm. A guide wire is initially passed through the blood vessel to a position near or at the desired point of implantation. A self-expanding stent is typically provided restrained in a radially compressed configuration within a sheath or catheter. A balloon expandable stent typically does not need to be restrained and is therefore merely positioned within a sheath or catheter. The stent and associated catheter are typically configured to be received over the guide wire and positioned over the guide wire at the desired location. When the catheter has been properly positioned within the blood vessel, the catheter is manipulated to cause the stent to be removed from the surrounding sheath or catheter in which it is restrained or positioned. The stent then expands or is expanded. Stent expansion may be effected by spring elasticity, balloon expansion, or by the self-expansion of a thermally or stress-induced return of a memory material to a pre-conditioned expanded configuration. At various points during implantation, various components of the catheter system are withdrawn and typically only the stent remains after implantation.

Stents need to be axially flexible for tracking through tortuous pathways of the human body. The use of bridges for connecting various sections of the stent has, in the past, satisfied this need to some extent. For example, bridges which are straight or curved in a zigzag manner permit flexion or extension of the stent. However, the surface area between stent components in stents incorporating bridges of this type is relatively small and the resulting gaps between stent components may become sites of restenosis. This is particularly frustrating where the stents have drug coatings designed to prevent restenosis.

It would be a significant advance in the art to provide connectors or bridges which will enable the stent to conform to tortuous body channels and which will track easily. In addition, the connectors should have sufficient surface areas to prevent gaps between stent components.

It would also be a significant advance if the stent could be manufactured economically and if inexpensive quality control were available.

Accordingly, it will be appreciated that there is a need for stents which address these and other needs and generally improve upon the stents now in the public domain. The present invention provides advantages over the prior art devices and solves other problems associated therewith.

### SUMMARY OF THE INVENTION

In preferred embodiments, the expandable stent of the present invention can either be self-expandable upon deployment or can be expanded by enlarging an expandable balloon positioned within the stent. The preferred stent includes a plurality of modules which are interconnected to each other by a series of flexible interconnecting cells.

The flexible interconnecting cells preferably have the form of “closed cells”. However, in contrast to the closed cell design of stents, the flexible interconnecting cells do not expand radially upon expansion of the stent during deployment. Rather, they expand or contract axially, depending upon whether the stent needs to conform to an outer convex curve or an outer concave curve in the tortuous pathways of the body channel in which the stent is deployed. Thus, where the stent needs to conform to a concavity in the channel, the flexible cell is compressed axially like an accordion. Where the stent needs to conform to a convexity, the flexible cell is expanded axially.

Typical closed cell designs for the flexible interconnecting cells or interconnects of the present invention include curved as well as angular embodiments. For example, lobed and saw-toothed configurations can be used. A particularly preferred embodiment is an axially oriented dual lobed configuration. Quadri-lobed configurations can also be used. Various other curved and angular shapes can be used as well, provided they can be expanded and compressed in the axial but not the radial direction.

The stents of the present invention are expandable, typically, for example, by enlarging an expandable balloon positioned within the stent, which preferably comprises a plurality of expandable modules, such as a plurality of expandable ring structures. The ring structures are joined to one another by means of flexible interconnecting cells or interconnects, as described above. The stents feature an absence of potential tissue snagging structures. The ring structures can articulate with respect to one another, which enables the stent to pass through otherwise tortuous passageways with many “sharp” turns or twists. The stents and ring structures of the stents are also characterized by relatively low surface areas compared to the surface area of a simple cylinder of similar dimensions

The stents of the present invention are efficiently and easily produced using laser etching or chemical etching techniques and are amenable to good quality control at a relatively low cost. Moreover, the stents of the present invention, in certain embodiments, provide little or no end-to-end shortening upon expansion, which may be especially desirable during certain procedures.

These and various other advantages and features of novelty which characterize the present invention are pointed out with particularity in the claims annexed hereto and forming a part hereof. However, for a better understanding of the present invention, its advantages and other objects obtained by its use, reference should be made to the drawings, which form a further part hereof, and to the accompanying descriptive matter, in which there are illustrated and described preferred embodiments of the present invention.

### BRIEF DESCRIPTION OF THE DRAWINGS

In the drawings, in which like reference numbers indicate corresponding parts throughout the several views;

FIG. 1 is a perspective view of a first embodiment of an expandable stent according to the present invention:

FIG. 2 is a side view of the embodiment of FIG. 1, temporarily mounted upon a balloon catheter and shown in close association with a longitudinal section of a stenosis in an artery about to be treated;

FIG. 3 is a side view of the embodiment of FIG. 1, following inflation of the balloon catheter inflated to deform and expand the expandable stent and treat the stenotic condition shown in longitudinal section;

FIG. 4 is a partial plan view of an enlarged portion of a first preferred embodiment of the present invention, showing the stent in a planar configuration prior to rolling into the cylindrical form in which it is used;

FIG. 5 is a a partial plan view of an enlarged portion of another preferred embodiment of the present invention, showing the stent in a planar configuration prior to rolling into the cylindrical form in which it is used;

FIG. 6 is an enlarged front elevation of the embodiment of FIG. 1 and FIG. 4, showing the stent conforming to a curve in the body passage in which the stent is deployed;

FIGS. 7A,7B, 7C, 8A, 8B, 8C, 9A, 9B, 9C, 10A, 10B, 10C, 11A, 11B, 11C, 12A, 12B, and 12C are enlarged plan views of several typical configurations of the interconnects used in the stents of the present invention, showing compressed and expanded states of each of six interconnect configurations;

FIG. 13 is an enlarged plan view of another configuration of interconnect;

FIG. 14 is an enlarged plan view of another configuration of interconnect;

FIG. 15 is an enlarged plan view of another configuration of interconnect; and

FIG. 16 is an enlarged plan view of another configuration of interconnect used in the stents of the present invention.

### DETAILED DESCRIPTION OF THE INVENTION

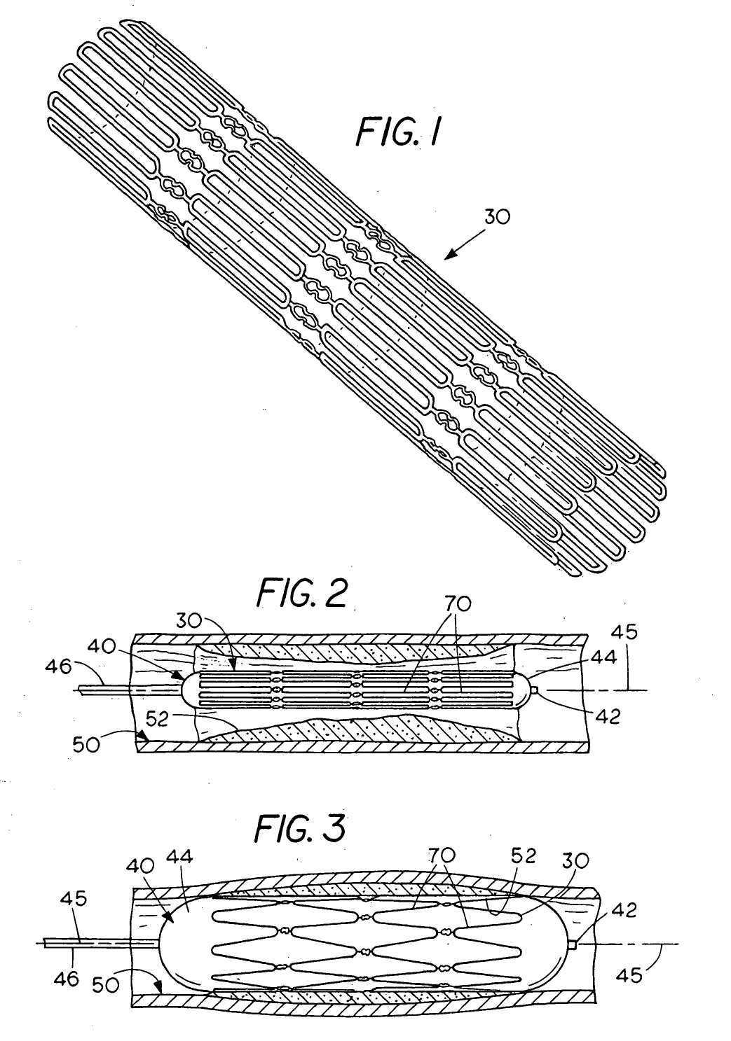

An expandable stent 30 according to the present invention is shown in FIG. 1.

In FIG. 2 the stent 30 is shown as being temporarily fitted upon or generally coaxial with a balloon catheter 40, having a distal end 42, an expandable balloon 44, and a catheter shaft 46. The stent 30 is also shown closely associated within a portion of an artery 50, which is partially occluded by a stenosis 52.

As shown schematically in FIG. 3, once the stent 30 is appropriately located in the lumen of the artery 50, preferably spanning the stenosis 52, the stent 30 can be expanded outward radially by inflating the balloon 44 of the balloon catheter 40. Inflation of balloon 44 is accomplished by application of fluid pressure to its interior by the cardiologist, acting at the proximal end (not shown) of catheter 40 in a manner which is well known in the art. As balloon 44 expands, stent 30 is also expanded outward radially. As the expansion continues, the stent 30 and balloon 44 contact and begin to alter the shape of the stenosis 52. Such expansion is continued until the stenosis 52 is reformed to a more desirable shape and size, i.e. more nearly cylindrical, such that patency is restored in the artery 50. The stent 30, shown in FIGS. 1, 2, and 3 is especially flexible longitudinally. This flexibility makes it considerably easier to introduce into coronary arteries having many turns and sharp bends. Furthermore, tissue prolapse is minimized with the present stent 30.

The relatively narrow, initial radius of the stent 30 positioned coaxially about axis 45 of the balloon 44 and not yet expanded to contact the stenosis 52 of artery 50 is shown schematically in FIG. 2. As shown in FIG. 3, the balloon 44 can be inflated to expand the stent 30 and force the stenosis 52 back against the wall of artery 50. Next, the fluid pressure on the balloon 44 can be relieved and reduced. The balloon 44 will contract radially toward axis 45 so that it can be easily withdrawn. The expandable stent 30, however, generally retains the expanded radius and does not contract, because it is preferably made of a low memory material such as stainless steel. In turn, the retained expanded condition of the stent 30 serves to hold the stenosis 52 out of the channel of the artery 50 and to restore patency to the artery 50. Because the stent 30 remains expanded but the balloon 44 contracts, withdrawal of the balloon 44 and the balloon catheter 40 is generally straightforward. Even after the balloon catheter 40 is withdrawn from the patient, patency remains in the artery 50 and more appropriate circulation is possible for the tissues served by the treated artery 50. The stent 30 remains as a support or scaffolding for the artery 50 and may also inhibit tissue prolapse and reformation of the stenosis 52.

In alternate embodiments, the present invention includes a method of making a stent. The method includes providing a segment of cylindrical walled material from which the stent will be made. Depending upon the type of stent to be made, any of the materials herein discussed or other materials that are well known in the art may be used, depending upon the particular characteristics desired. The stent is prepared by removal of material from the cylindrical wall, which material will not be part of the stent to be formed. This may occur by mechanically cutting away material. Preferably, however, the cutting or material removal is automated. A computer aided laser-cutting device is one option. A computer aided water-jet cutting device is another option. In each case, software that guides the cutting tool will assure that only the material, which is intended to be removed, is in fact removed. Another removal technique is chemical etching of the cylinder wall. The portion of the cylinder to be retained as a part of the stent is protected from exposure to the chemical etching process. For example, in the case of a metallic stent, an etching agent might be one of a number of acids, which are well known in the art. A chemically protective agent, for example, a hydrophobic coating, such as a wax, may be applied over the entire exterior surface of the cylinder. Next, the protective coating is removed mechanically using a computer aided water jet cutting device, or the like, where etching is desired. If greater surface thickness is desired, wider areas need to be protected. If thinner surface thickness is desired, then narrower areas are protected. Alternatively, other means of selectively applying protective coatings, for example, photographically based methods, which are well known in the etching arts, may be used. Finally, the partially protected cylinder is immersed in an acid bath. Etching occurs throughout the interior cylinder surface but only at selected portions of the exterior surface. When the etching has proceeded to the extent that the etching from the exterior and interior surfaces has fully removed appropriate portions of the cylinder, the piece is removed from the acid. Next, the protective coating is removed. If the coating is wax, the wax may be removed by heating or by a wax solvent, which does not further affect the metal. Chemical etching is a suitable production method for low volume production. Higher volume production is believed to be more suitably achieved through the use of computer aided laser etching. The availability of using wider or narrower surface thicknesses, as well as different tubing wall thicknesses is considered an important means of obtaining stiffness or easier deformability in the desired devices of the present invention. Generally, thin walled tubing is believed to be preferable, but not absolutely required.

An alternate material from which expandable stents of this invention may be prepared is, without limit, stainless steel, particularly type 316 stainless steel, more preferably type 316 L or 316 Lvm stainless steel, but gold, platinum, tantalum, silver and the like are also believed to be suitable. Desirable features of the material selected are deformability and the ability to hold the shape once deformed. It is also desirable that the stent 30 be made from radiopaque materials. Stents made of stainless steel which have a thickness of 0.005 inch are generally radiopaque. However, stents having lesser thicknesses, such as stents made specifically for use in coronary arteries which often requires thicknesses less than 0.005 inch (often, for example, about 0.003 inch) need to be coated with a radiopaque material such as 24 carat gold to a thickness of about 0.0002 inch. In addition, other coatings including specific functional agents may also be employed to address issues such as blood clotting (e.g. heparin and the like) or reduction in the amount of intimal hyperplasia and resulting restenosis (e.g. cytotoxic drugs, gene therapy agents and the like). Methods to coat metal prostheses to make them radiopaque or to minimize the risks due to blood clotting are well known in the art and any of these methods and the devices resulting from the use of these methods are all envisioned within the scope of the present invention.

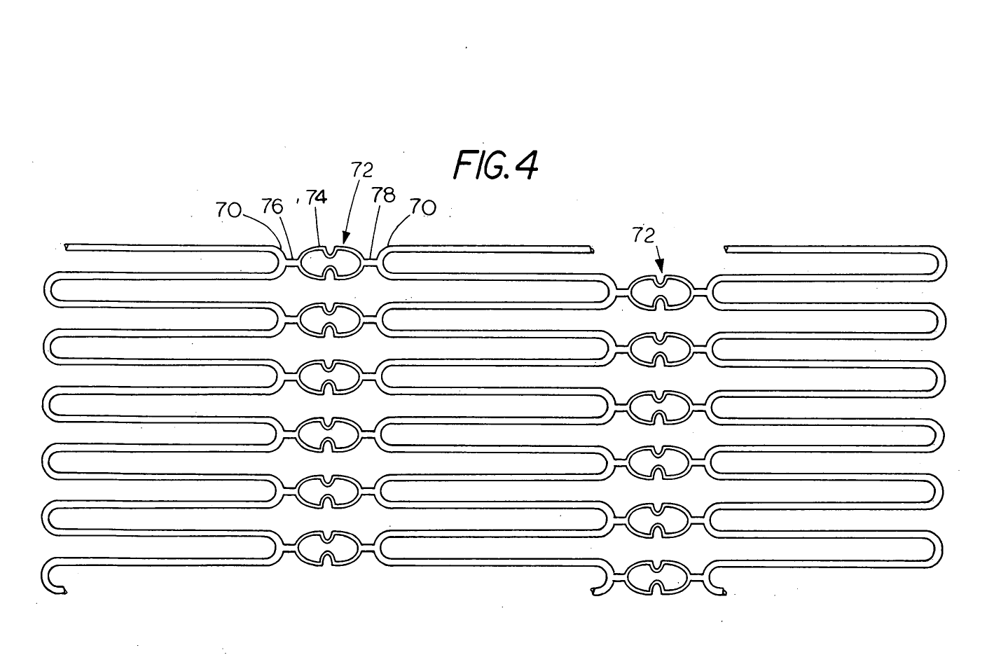

FIG. 4 illustrates a preferred embodiment of the present invention in a pre-formed stage prior to forming into the cylindrical configuration of the stent. Modules 70 are interconnected by means of flexible interconnecting cells or interconnects 72. Each of the interconnects 72 has a flexible body 74 and connectors 76 and 78, one at each end of body 74. Interconnects 72 are configured to expand or contract in the axial direction, but not in the radial direction.

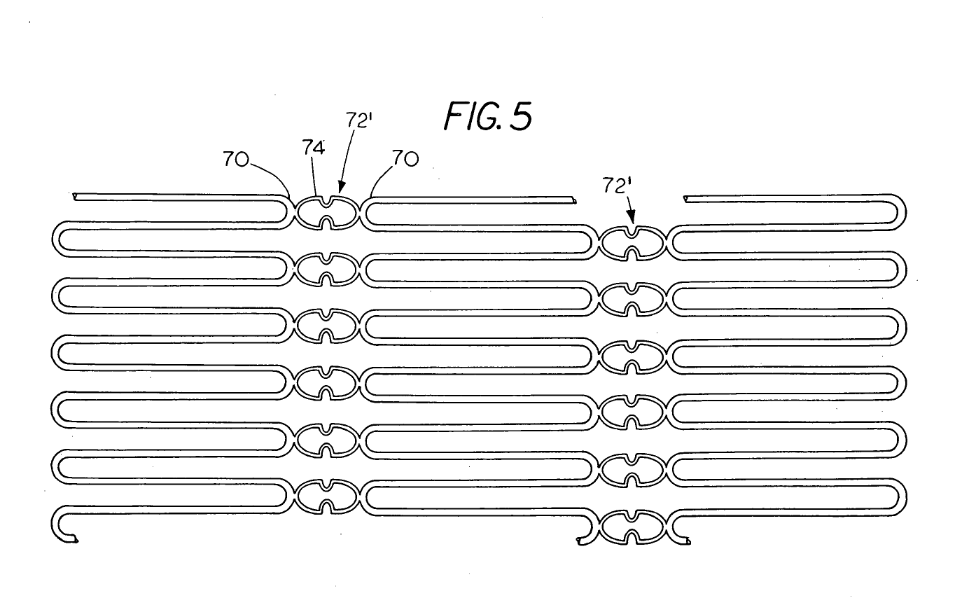

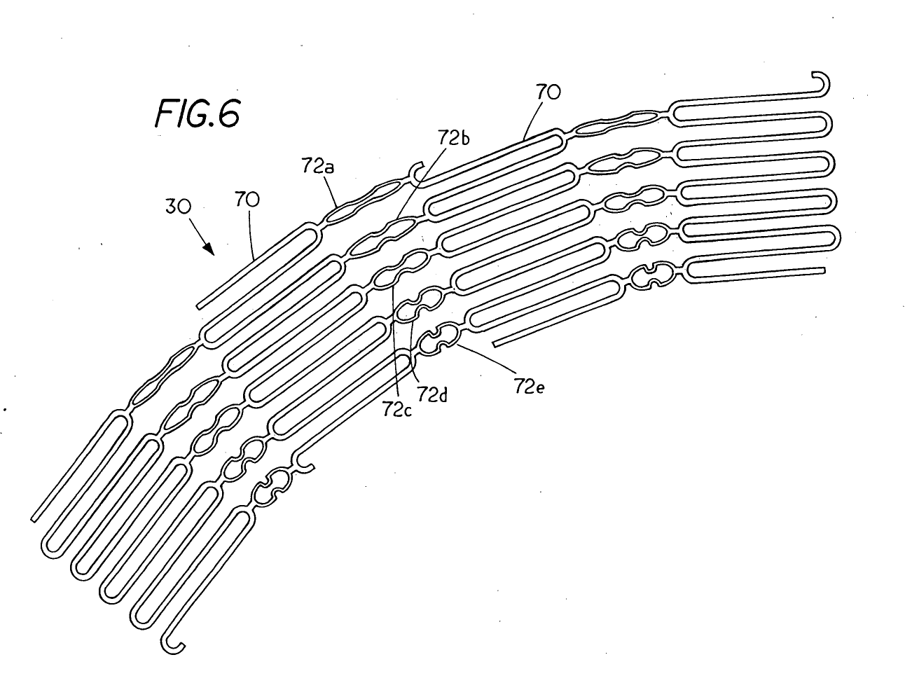

FIG. 5 illustrates another preferred embodiment of the present invention in a pre-formed stage prior to forming into the cylindrical configuration of the stent. Modules 70 are interconnected by means of flexible interconnecting cells or interconnects 72′. Each of the interconnects 72′ has a flexible body 74, but no connectors corresponding to the connectors 76 and 78 of FIG. 4. That is, the flexible body 74 abuts modules 70. Interconnects 72′ are configured to expand and contract in the axial direction, but not in the radial direction.

FIG. 6 shows how the flexible interconnecting cells or interconnects of the present invention enable the stent to navigate tortuous curves and turns encountered in the body passageways in which the stents are deployed. A convexity encountered by stent 30 in a body passageway causes interconnects 72a and 72b to expand in the axial direction from the normal configuration of interconnect 72c. Interconnect 72a is shown expanded more than interconnect 72b. Interconnects 72d and 72e contract in the axial direction as a result of encountering a concavity in a body passageway. Intercconnect 72e is shown contracted more than interconnect 72d.

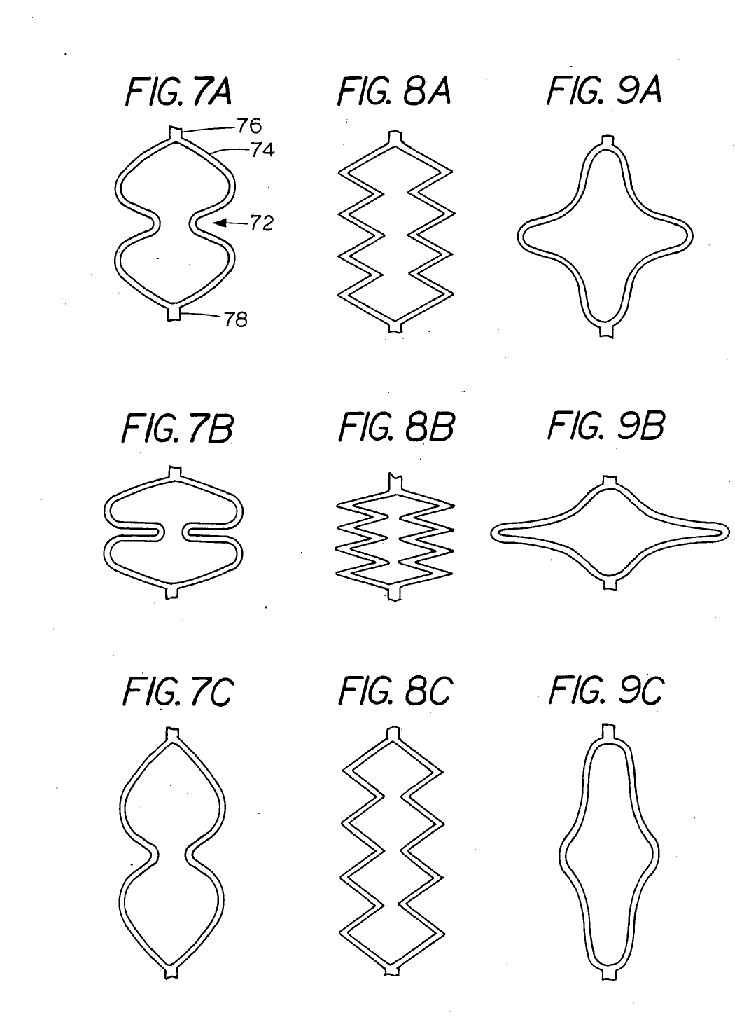

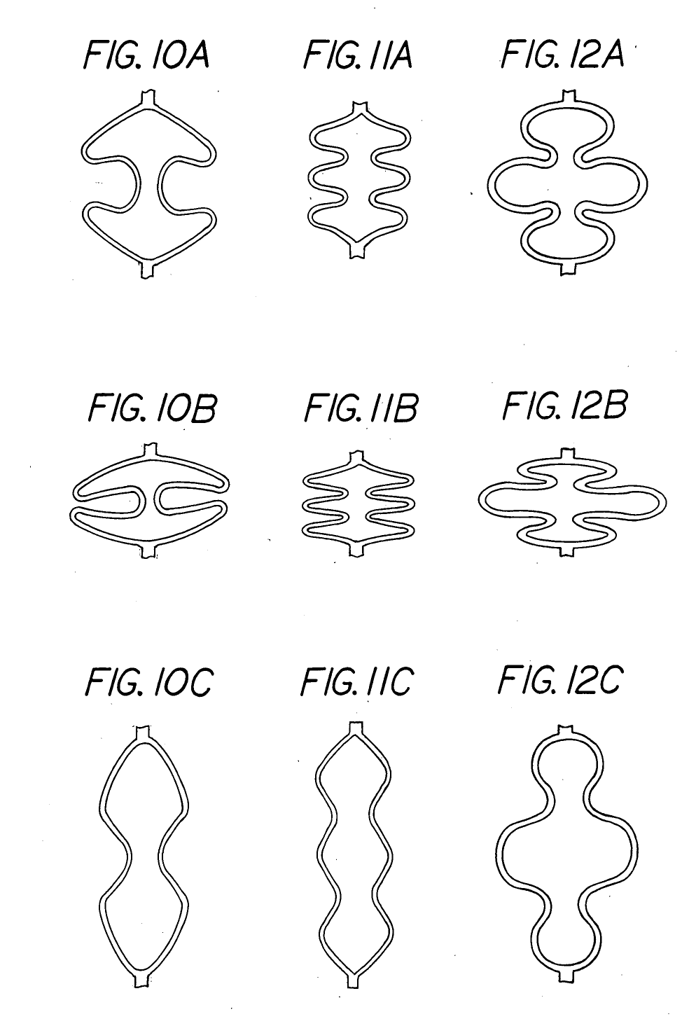

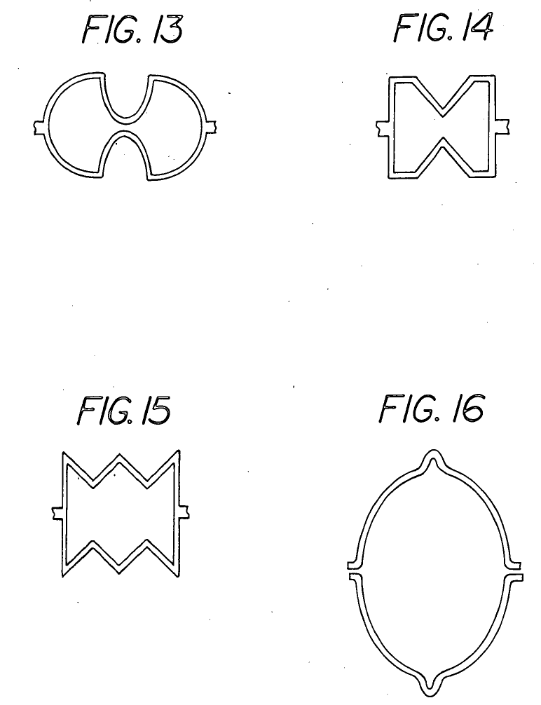

As pointed out above, the flexible interconnecting cells or interconnects 72 or 72′ of the present invention may be provided with flexible bodies 74 in various configurations. Several examples of such configurations are depicted diagrammatically in FIGS. 7A-7C, 8A-8C, 9A-9C, 10A-10C, 11A-11C, 12A-12C, 13, 14, 15, and 16. Each body configuration of interconnect has an indentation or lobe at the axial midpoint of flexible body 74 to enable the interconnect to expand or contract in the axial direction. Furthermore, as best shown in FIGS. 1, 4 5, and 6, these interconnect configurations provide sufficient areas of contact to enable coating with various medicinal and other functional agents as pointed out above. In addition, these configurations contribute mechanical strength to the stents in which they are employed.

The flexible interconnects of the present invention are designed to expand and contract in the axial direction to facilitate navigation through tortuous twists and turns in body passageways. Contracted states of the configurations of FIGS. 7A, 8A, 9A, 10A, 11A, and 12A are depicted diagrammatically in FIGS. 7B, 8B, 9B, 10B, 11B, and 12B. Expanded states of the configurations of FIGS. 7A, 8A, 9A, 10A, 11A, and 12A are depicted diagrammatically in FIGS. 7C, 8C, 9C, 10C, 11C, and 12C.

It is understood that even though numerous characteristics and advantages of various embodiments of the present invention have been set forth in the foregoing description, together with details of the structure and function of various embodiments of the invention, this disclosure is illustrative only and changes may be made in detail, especially in matters of shape, size and arrangement of parts, within the principles of the present invention, to the full extent indicated by the broad general meaning of the terms in which the appended claims are expressed.

What is claimed is:

1. An expandable stent comprising: a plurality of modules interconnected axially by a plurality of interconnects, each of the plurality of interconnects being in the form of a closed cell which can expand or contract axially.

2. The expandable stent of claim 1, wherein the interconnects are adapted to contract axially upon encountering a concavity in a passageway in which the stent is deployed and to expand axially upon encountering a convexity therein.

3. The expandable stent of claim 1, wherein the interconnect comprises a flexible body and connectors attached to the flexible body for connecting adjacent modules.

4. The expandable stent of claim 1, wherein the interconnect comprises a flexible body which abuts adjacent modules of the stent.

5. The expandable stent of claim 1, wherein the flexible interconnect has a curved periphery.

6. The expandable stent of claim 1, wherein the flexible interconnect has an angular periphery.

7. The expandable stent of claim 5, wherein the curved periphery is lobed.

8. The expandable stent of claim 7, wherein the lobed periphery is a dual lobed periphery.

9. The expandable stent of claim 7, wherein the lobed periphery is a quadri-lobed periphery.

10. The expandable stent of claim 6, wherein the angular periphery is saw-toothed.

11. The expandable stent of claim 3, wherein the flexible body of the interconnect has a curved periphery.

12. The expandable stent of claim 3, wherein the flexible body of the interconnect has an angular periphery.

13. The expandable stent of claim 11, wherein the curved periphery is lobed.

14. The expandable stent of claim 13, wherein the lobed periphery is a dual lobed periphery.

15. The expandable stent of claim 13, wherein the lobed periphery is a quadri-lobed periphery.

16. The expandable stent of claim 12, wherein the angular periphery is saw-toothed.

17. The expandable stent of claim 1, wherein the stent is self-expanding.

18. The expandable stent of claim 1, wherein the stent is expandable by inflating a balloon positioned within the stent.
